(19) United States
(12) Patent Application Publication
(10) Pub. No.: US 2025/0215483 A1
(43) Pub. Date: Jul. 3, 2025

(54) DEVICE, APPARATUS AND METHOD FOR DETECTING NUCLEIC ACIDS

(71) Applicant: Siemens Healthcare Diagnostics Inc., Tarrytown, NY (US)

(72) Inventors: Ramya V M, Bangalore (IN); Nivedita MITRA, Bangalore (IN); Divya KHANDIGE, Bangalore (IN)

(21) Appl. No.: 18/844,080

(22) PCT Filed: Mar. 8, 2023
(86) PCT No.: PCT/US2023/063989

§ 371 Date: Sep. 5, 2024

(30) Foreign Application Priority Data
Mar. 9, 2022 (IN) 202241012844

(51) Int. Cl. C12Q 1/6834

(52) U.S. Cl. CPC C12Q 1/6834

(57) ABSTRACT

The present disclosure refers to a homogeneous method, apparatus and device for detection of target nucleic acids in a sample. In one aspect, the device includes a magnetic bead. Further, the device includes one or more oligonucleotides bound to the magnetic bead. Additionally, the device includes at least one reporter molecule linked to the one or more oligonucleotides.

### CROSS-REFERENCE TO RELATED APPLICATIONS

This application claims priority or the benefit under 35 U.S.C. § 119 of IN provisional patent application No. 202241012844 filed on 9 Mar. 2022, the contents of which are fully incorporated herein by reference.

### FIELD

The present disclosure relates to the field of analysis of a biological sample and more particularly to the field of detection of analytes-of-interest such as nucleic acids in a biological sample.

### BACKGROUND

Sensitive and rapid detection of analytes-of-interest such as nucleic acids plays an important role in disease management. Early detection of infectious pathogens in a sample is enabled by a sensitive assay. This aids an early treatment of the disease and prevents disease spread. To ensure the reach of an assay is maximized, it is important the assay be performed with minimal instrumentation and sample pre-processing.

Current methods of nucleic acid detection may include reverse transcriptase polymerase chain reaction (RT-PCR) and other isothermal amplification methods such as Loop-mediated isothermal amplification (LAMP). The efficiency of such methods is better when purified nucleic acid sample is used. Therefore, such methods are complex and time consuming. Additionally, RT-PCR and LAMP can be instrument intensive and need a lot of manual intervention for optimal performance.

Therefore, there is a continuing need for detecting analytes-of-interest such as nucleic acids in a biological sample which is quick or requires minimal instrumentation and sample pre-processing. Moreover, there is a need in the art for new and improved diagnostic assay reagents that overcome defects and disadvantages of the prior art. It is to such reagents, kits, and microfluidic apparatuses, as well as methods of producing and using same, that the present disclosure is directed.

### SUMMARY

Methods, devices and apparatuses for analyte-of-interest detection, such as nucleic acid detection (e.g., DNA or RNA) are provided herein. In embodiments, the presence of certain DNA or RNA is indicative of the presence of a pathogen or diseased state of a subject.

In some embodiments, the present disclosure includes a device for detection of target nucleic acids in a sample. In one aspect of the present disclosure, the device includes a magnetic bead and one or more oligonucleotides bound to the magnetic bead. Additionally, in embodiments, the device includes at least one reporter molecule or label linked to the one or more oligonucleotides. In embodiments, the one or more oligonucleotides are preselected in length, and chemical composition. In embodiments, the one or more oligonucleotides are double stranded and include a loop structure, wherein the loop structure includes a first single stranded nucleic acid segment and a second single stranded nucleic acid segment, wherein one of the first or second single stranded nucleic acid segments is characterized as an analyte specific binding segment.

In another aspect of the disclosure, a method for detection of nucleic acids in a sample is disclosed. In embodiments, the method includes introducing a sample, such as a biological sample, to a device, wherein the device includes a magnetic bead, one or more oligonucleotides bound to the magnetic bead and at least one reporter molecule or label linked to the one or more oligonucleotides. In embodiments, the magnetic bead in bound to a first end of the oligonucleotide and the reporter is bound to a second end of the same oligonucleotide. Additionally, in embodiments, a method of the present disclosure includes incubating the sample such as a biological sample and the device to form a complex, wherein the complex includes one or more target nucleic acids from the biological sample bound to the one or more oligonucleotides in the device. In embodiments, the one or more nucleotides include at least one portion or segment of nucleotides complimentary to the target nucleic acids present or sought in the biological sample. In embodiments, the one or more oligonucleotides are double stranded and include a loop structure, wherein the loop structure includes a first single stranded nucleic acid segment and a second single stranded nucleic acid segment, wherein one of the first or second single stranded nucleic acid segments is characterized as an analyte specific binding segment and complimentary or substantially complementary to a target nucleic acid or analyte-of-interest or portion thereof.

Furthermore, in embodiments, a method of the present disclosure includes separating the complex from the device and detecting the target nucleic acids in the sample from the separated complex.

In yet another aspect, an apparatus such as a microfluidic device for detection of target nucleic acids in a sample is disclosed. The apparatus includes a first chamber configured to receive the sample, wherein the first chamber includes a buffer, and a plurality of devices of the present disclosure. In embodiments, the device includes a magnetic bead, one or more oligonucleotides bound to the magnetic bead and at least one reporter molecule linked to the one or more oligonucleotides. In embodiments, the one or more oligonucleotides are double stranded and include a loop structure, wherein the loop structure includes a first single stranded nucleic acid segment and a second single stranded nucleic acid segment, wherein one of the first or second single stranded nucleic acid segments is characterized as an analyte specific binding segment and complimentary or substantially complementary to a target nucleic acid. In embodiments, the analyte specific binding segment binds to the target nucleic acid in the biological sample.

Further, in embodiments, the apparatus includes a second chamber configured to receive the sample and the plurality of the device, wherein the second chamber includes a substrate capable of reacting with a reporter molecule linked to the one or more oligonucleotides in the device.

In embodiments, the present disclosure includes a kit including one or more devices of the present disclosure, one or more apparatuses or microfluidic devices of the present disclosure. Optionally, kit embodiments may include a swab, fluid or buffer solution.

This summary is provided to introduce a selection of concepts in a simplified form that are further described below in the following description. It is not intended to identify features or essential features of the claimed subject matter. Furthermore, the claimed subject matter is not limited to implementations that solve any or all disadvantages noted in any part of this disclosure.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 illustrates a device for detection of target nucleic acids in a sample, according to an embodiment of the present disclosure.

FIG. 2 illustrates a device for detection of target nucleic acids in a sample, according to another embodiment of the present disclosure.

FIG. 3 illustrates an apparatus for detection of target nucleic acids in a sample, according to an embodiment.

FIG. 4 illustrates a method of detection of target nucleic acids in sample, according to an embodiment.

FIG. 5 illustrates a method of detection of target nucleic acids in a sample, according to another embodiment.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION OF CERTAIN EMBODIMENTS

Hereinafter, embodiments for carrying out the present invention are described in detail. In embodiments, the present disclosure includes methods, devices and apparatuses for analyte-of-interest detection, such as nucleic acid detection (e.g., DNA or RNA) indicative of one or more pathogens in a biological sample or diseased state of a subject. In some embodiments, the present disclosure includes a device for detection of target nucleic acids in a sample such as a device including a magnetic bead and one or more oligonucleotides bound to the magnetic bead. In embodiments, the device includes at least one reporter molecule or label linked to the one or more oligonucleotides. In embodiments, the one or more oligonucleotides are preselected in length, and chemical composition. In embodiments, the one or more oligonucleotides are double stranded and include a loop structure, wherein the loop structure includes a first single stranded nucleic acid segment and a second single stranded nucleic acid segment, wherein one of the first or second single stranded nucleic acid segments is characterized as an analyte specific binding segment, and complimentary or substantially complementary to a target nucleic acid. In embodiments, the target nucleic acid is DNA or RNA indicative of the presence of one or more pathogens in a biological sample or a diseased state of a subject.

Embodiments of the present disclosure advantageously provide an improved method, apparatus and/or device for detecting one or more analytes-of-interest such as nucleic acid molecules of interest. In embodiments, the nucleic acid molecules of interest indicate the presence of one or more pathogenic species in a biological sample or the diseased state of a subject. Embodiments of the present disclosure advantageously improve the time, cost, and efficiency of detecting analytes-of-interest such as nucleic acids in a biological sample. Embodiments of the present disclosure advantageously improve the time, cost, and efficiency of detecting nucleic acids characterized as biomarkers indicative of a diseased or deficient state of a subject.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. In embodiments, standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

### Definitions

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A. B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, vaginal discharge, fecal, pleural fluid, nasopharyngeal fluid, combinations thereof, and the like.

The term “specific binding partner,” as used in particular (but not by way of limitation) herein in the term “target analyte-specific binding partner,” will be understood to refer to any molecule capable of specifically associating with the target analyte. For example, but not by way of limitation, the binding partner may be a DNA segment, an RNA segment, a single stranded nucleic acid segment, combinations or derivatives thereof, as well as any other molecules capable of specific binding to the target analyte of interest.

An “analyte” is a nucleic acid macromolecule that is capable of being recognized by an analyte-specific binding partner. In embodiments, an analyte refers to a nucleic acid macromolecule that is capable of being recognized by an analyte-specific binding partner such as (but not limited to) a DNA or RNA segment, strand or oligomer or portion thereof that is complimentary or substantially complimentary to the analyte such that it is able to bind thereto.

As used herein, a “nucleoside” is a base-sugar combination and “nucleotides” are nucleosides that further include a phosphate group covalently linked to the sugar portion of the nucleoside. As used herein, a nucleoside with a modified sugar residue is any nucleoside wherein the 2′-deoxyribose sugar has been substituted with a chemically modified sugar moiety. In the context of the present disclosure, the chemically modified sugar moieties include, but are not limited to, 2′-O-methoxyethyl, 2′-fluoro, 2′-dimethylaminooxyethoxy, 2′-dimethylaminoethoxyethoxy, 2′-guanidinium, 2′-O-guanidinium ethyl, 2′-carbamate, 2′-aminooxy, 2′-acetamido and locked nucleic acid.

As used herein, “targeting” or “targeted to” refer to the process of designing or preselecting an oligomeric compound or oligonucleotide such that the oligomeric compound or oligonucleotide or a portion thereof hybridizes with a selected nucleic acid molecule or region of a nucleic acid molecule such as from an analyte-of-interest.

As used herein, “hybridization” means the pairing of complementary strands of oligomeric compounds. In the context of the present disclosure, an oligomeric compound is specifically hybridizable when there is a sufficient degree of complementarity to avoid non-specific binding of the oligomeric compound to non-target nucleic acid sequences. In embodiments, one of skill in the art will be able to determine when an oligomeric compound is specifically hybridizable. In some embodiments, the term “hybridization” refers to the formation of complexes (also called duplexes or hybrids) between nucleotide sequences which are sufficiently complementary to form complexes via Watson-Crick base pairing or non-canonical base pairing. It will be appreciated that hybridizing sequences need not have perfect complementary to provide stable hybrids. In many situations, stable hybrids will form where fewer than about 10% of the bases are mismatches. Accordingly, as used herein, the term “complementary” refers to a nucleic acid molecule that forms a stable duplex with its complement under particular conditions, generally where there is about 90% or greater homology (e.g., about 95% or greater, about 98% or greater, or about 99% or greater homology). Those skilled in the art understand how to estimate and adjust the stringency of hybridization conditions such that sequences that have at least a desired level of complementarity will stably hybridize, while those having lower complementarity will not. For examples of hybridization conditions and parameters, see, for example, Sambrook et al., “Molecular Cloning: A Laboratory Manual”; 1989, Second Edition, Cold Spring Harbor Press: Plainview, N.Y. and Ausubel, “Current Protocols in Molecular Biology”, 1994, John Wiley & Sons: Secaucus, N.J. Complementarity between two nucleic acid molecules is said to be “complete”, “total” or “perfect” if all the nucleic acid's bases are matched, and is said to be “partial” otherwise.

The terms “labeled” and “labeled with a detectable agent (or moiety)” are used herein interchangeably to specify that an entity (e.g., a target sequence) can be visualized, e.g., directly or following hybridization to another entity that comprises a detectable agent or moiety. Preferably, the detectable agent or moiety is selected such that it generates a signal which can be measured and whose intensity is related to (e.g., proportional to) the amount of the entity of interest (e.g., a target sequence). Methods for labeling nucleic acid molecules are well-known in the art. In some embodiments, labeled nucleic acids can be prepared by incorporation of, or conjugation to, a label that is directly or indirectly detectable by spectroscopic, photochemical, biochemical, immunochemical, electrical, optical, or chemical means.

In some embodiments, the term “oligonucleotide” is used herein to denote a polynucleotide that includes between about 5 and about 150 nucleotides, e.g., between about 10 and about 100 nucleotides, between about 15 and about 75 nucleotides, or between 15 and about about 50 nucleotides. Throughout the specification, whenever an oligonucleotide is represented by a sequence of letters (chosen, for example, from the four base letters: A, C, G, and T, which denote adenosine, cytidine, guanosine, and thymidine, respectively), the nucleotides are presented in 5′ to 3′ order from the left to the right. A “polynucleotide sequence” refers to the sequence of nucleotide monomers along the polymer. Unless denoted otherwise, whenever a polynucleotide sequence is represented, it will be understood that the nucleotides are in 5′ to 3′ orientation from left to right.

The term “nucleic acid” as used herein means a nucleobase polymer having a backbone of alternating sugar and phosphate units in DNA and RNA. In embodiments, “Nucleic acid” and “polynucleotide” are considered to be equivalent and interchangeable. Nucleic acids are commonly in the form of DNA or RNA. In some embodiments, the terms “nucleic acid”, “nucleic acid molecule”, “polynucleotide” or “oligonucleotide” are used herein interchangeably. They refer to polymers of nucleotide monomers or analogs thereof, such as deoxyribonucleic acid (DNA) and ribonucleic acid (RNA). The nucleotides may be genomic, synthetic or semi-synthetic in origin. Unless otherwise stated, the terms encompass nucleic acid-like structures with synthetic backbones, as well as amplification products. As will be appreciated by one skilled in the art, the length of these polymers (i.e., the number of nucleotides it contains) can vary widely, often depending on their intended function or use. Polynucleotides can be linear, branched linear, or circular molecules. In embodiments, polynucleotides also have associated counter ions, such as H⁺, NH₄⁺, trialkylammonium, Mg₂⁺. Na⁺ and the like. A polynucleotide may be composed entirely of deoxyribonucleotides, entirely of ribonucleotides, or chimeric mixtures thereof. Polynucleotides may be composed of internucleotide nucleobase and sugar analogs.

The term “3” refers to a region or position in a polynucleotide or oligonucleotide 3′ (i.e., downstream) from another region or position in the same polynucleotide or oligonucleotide.

The term “5” refers to a region or position in a polynucleotide or oligonucleotide 5′ (i.e., upstream) from another region or position in the same polynucleotide or oligonucleotide.

The terms “3′ end” and “3′ terminus”, as used herein in reference to a nucleic acid molecule, refer to the end of the nucleic acid which contains a free hydroxyl group attached to 3′ carbon of the terminal pentose sugar. In some embodiments of the present disclosure, targets are tagged at their 3′ terminus.

The term “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar. In some embodiments of the present disclosure, targets are tagged at their 5′ terminus.

The term “isolated”, as used herein, means a target, sample, polynucleotide, complex, nucleic acid or oligonucleotide, which by virtue of its origin or manipulation, is separated from at least some of the components with which it is naturally associated or with which it is associated when initially obtained.

As used herein, a support (also referred to as a matrix support, a matrix, an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein may be particulate or may be in the form of a continuous surface, such as a microtiter dish or well, a glass slide, a silicon chip, a nitrocellulose sheet, nylon mesh, or other such materials. When particulate, typically the particles have at least one dimension in the 5-100 μm range or smaller. Such particles, referred collectively herein as “beads”, are often, but not necessarily, spherical. Such reference, however, does not constrain the geometry of the matrix, which may be any shape, including random shapes, needles, fibers, and elongated. Roughly spherical “beads”, particularly microspheres that can be used in the liquid phase, are also contemplated. The “beads” may include additional components, such as magnetic or paramagnetic particles (see, e.g., Dynabeads® (Dynal, Oslo, Norway)) for separation using magnets, as long as the additional components do not interfere with the methods and analyses herein. As used herein, matrix or support particles refers to matrix materials that are in the form of discrete particles. The particles can have any shape and dimensions, but typically have at least one dimension that is 100 mm or less, 50 mm or less, 10 mm or less, 5 mm or less, 4 mm or less, 3 mm or less, 2 mm or less, 1 mm or less. 900 μm or less, 800 μm or less, 700 μm or less, 600 μm or less, 500 μm or less, 400 μm or less, 300 μm or less, 200 μm or less, 100 μm or less, 50 μm or less, 40 μm or less, 30 μm or less, 20 μm or less, 10 μm or less, 5 μm or less, 4 μm or less, 3 μm or less, 2 μm or less, 1 μm or less, 900 nm or less, 800 nm or less, 700 nm or less, 600 nm or less, 500 nm or less, 400 nm or less, 300 nm or less, 200 nm or less, 100 nm or less, 50 nm or less, 40 nm or less, 30 nm or less, 25 nm or less, and 10 nm or less. The particles typically have a size that is 100 mm³ or less, 50 mm³ or less, 10 mm³ or less, and 5 mm³ or less, 4 mm³ or less, 3 mm³ or less, 2 mm³ or less, and 1 mm³ or less, 900 μm³ or less, 800 μm³ or less, 700 μm³ or less, 600 μm³ or less, 500 μm³ or less, 400 μm³ or less, 300 μm³ or less, 200 μm³ or less, 100 μm³ or less, 50 μm³ or less, 40 μm³ or less, 30 μm³ or less, 20 μm³ or less, 10 μm³ or less, 5 μm³ or less, 4 μm³ or less, 3 μm³ or less, 2 μm³ or less, 1 μm³ or less, 900 nm³ or less, 800 nm³ or less, 700 nm³ or less, 600 nm³ or less, 500 nm³ or less, 400 nm³ or less, 300 nm³ or less, 200 nm³ or less, 100 nm³ or less, 50 nm³ or less, 40 nm³ or less, 30 nm³ or less, 20 nm³ or less, 10 nm³ or less, 5 nm³ and may be on the order of cubic nanometers; typically the particles have a diameter of about 1.5 microns and less than 15 microns, such as about 4-6 microns. In embodiments, such particles are collectively called “beads.”

As used herein, “substrate” refers to an insoluble support that can provide a surface on which or over which a reaction may be conducted and/or a reaction product can be retained at an identifiable locus. Support can be fabricated from virtually any insoluble or solid material. For example, silicon, silica gel, glass (e.g. controlled-pore glass (CPG)), nylon, Wang resin, Merrifield resin, Sephadex®, Sepharose®, cellulose, a metal surface (e.g., steel, gold, silver, aluminum, and copper), a plastic material (e.g., polyethylene, polypropylene, polyamide, polyester, polyvinylidenedifluoride (PVDF)). Exemplary substrates include, but are not limited to flat supports such as glass fiber filters, silicon surfaces, glass surfaces, metal surfaces (steel, gold, silver, aluminum, and copper), and plastic materials. In embodiments, a solid support is in any desired form suitable for mounting on a cartridge base, including, but not limited to: a plate, membrane, wafer, a wafer with pits and other geometries and forms known to those of skill in the art. Exemplary supports are flat surfaces designed to receive or link samples at discrete loci, such as flat surfaces with hydrophobic regions surrounding hydrophilic loci for receiving, containing or binding a sample. Non-limiting examples of substrates are described in U.S. Pat. No. 8,088,573.

Turning now to the inventive concepts, certain non-limiting embodiments of the present disclosure are directed to a diagnostic reagent composition (such as, but not limited to, a diagnostic immunoassay reagent composition) for detection of a target analyte in a biological sample. In embodiments, the present disclosure includes a device for detection of target nucleic acids (such DNA or RNA indicating the presence of a pathogen or diseased state of a subject) in a sample, the device including: a magnetic bead; one or more oligonucleotides bound to the magnetic bead; and at least one reporter molecule or label linked to the one or more oligonucleotides. In embodiments, the one or more oligonucleotides include at least one portion of nucleotides complimentary or substantially complimentary to a target nucleic acid or preselected target nucleic acid present in the sample. In embodiments, the one or more oligonucleotides is a combination of a primary oligonucleotide linked to a secondary oligonucleotide, wherein at least one portion of the secondary oligonucleotide is complimentary to the primary oligonucleotide. In embodiments, the at least one reporter molecule is linked to the secondary oligonucleotide. In embodiments, the secondary oligonucleotide includes at least one portion of nucleotides complimentary to the target nucleic acids present in the sample or substantially complimentary to the target nucleic acids present in the sample. In embodiments, the reporter molecule is one of an enzyme, a fluorescent protein, or a quantum dot.

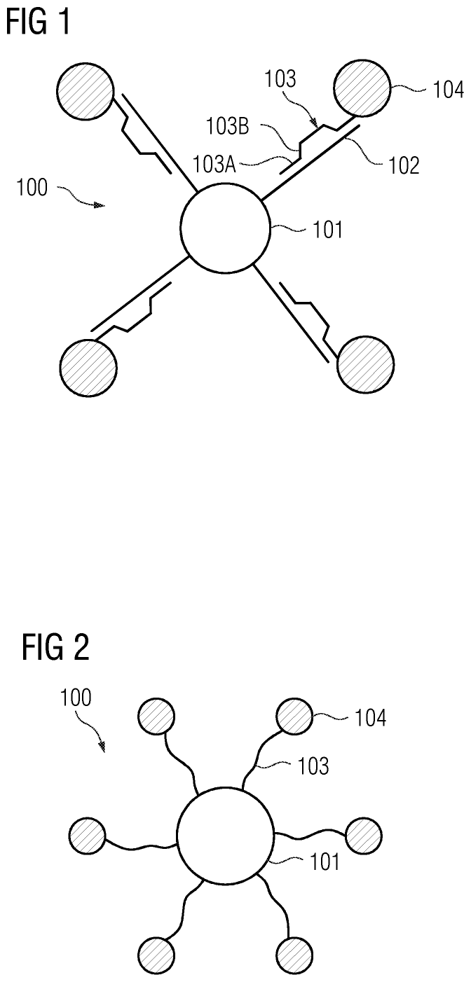

Referring now to FIG. 1, a device 100 for detecting or targeting nucleic acids in a sample, according to an embodiment of the present disclosure is shown. In embodiments, the device 100 includes a magnetic bead 101 to which one or more oligonucleotides 102, 103 are bound.

In embodiments, magnetic bead 101 is characterized being in the form of discrete particles. In embodiments, the particles can have any shape and dimensions, but typically have at least one dimension that is 100 mm or less, 50 mm or less, 10 mm or less, 5 mm or less, 4 mm or less, 3 mm or less, 2 mm or less, 1 mm or less, 900 μm or less, 800 μm or less, 700 μm or less, 600 μm or less, 500 μm or less, 400 μm or less, 300 μm or less, 200 μm or less, 100 μm or less, 50 μm or less, 40 μm or less, 30 μm or less, 20 μm or less, 10 μm or less, 5 μm or less, 4 μm or less, 3 μm or less, 2 μm or less, 1 μm or less, 900 nm or less, 800 nm or less, 700 nm or less, 600 nm or less, 500 nm or less, 400 nm or less, 300 nm or less, 200 nm or less, 100 nm or less, 50 nm or less, 40 nm or less, 30 nm or less, 25 nm or less, and 10 nm or less. The particles typically have a size that is 100 mm³ or less, 50 mm³ or less, 10 mm³ or less, and 5 mm³ or less, 4 mm³ or less, 3 mm³ or less, 2 mm³ or less, and 1 mm³ or less, 900 μm³ or less, 800 μm³ or less, 700 μm³ or less, 600 μm³ or less, 500 μm³ or less, 400 μm³ or less, 300 μm³ or less, 200 μm³ or less, 100 μm³ or less, 50 μm³ or less, 40 μm³ or less, 30 μm³ or less, 20 μm³ or less, 10 μm³ or less, 5 μm³ or less, 4 μm³ or less, 3 μm³ or less, 2 μm³ or less, 1 μm³ or less, 900 nm³ or less, 800 nm³ or less, 700 nm³ or less, 600 nm³ or less, 500 nm³ or less, 400 nm³ or less, 300 nm³ or less, 200 nm³ or less, 100 nm³ or less, 50 nm³ or less, 40 nm³ or less, 30 nm³ or less, 20 nm³ or less, 10 nm³ or less, 5 nm³ and may be on the order of cubic nanometers; typically the particles have a diameter of about 1.5 microns and less than 15 microns, such as about 4-6 microns. Such particles are collectively called “beads.” In embodiments, beads are characterized as magnetic or paramagnetic particles (see, e.g., Dynabeads® (Dynal, Oslo, Norway)) suitable for separation using magnets. In embodiments, the magnetic bead 101 is a solid, spherical structure which acts as a base for the oligonucleotides 102 to bind.

Still referring to FIG. 1, the oligonucleotides 102 may be bound to the magnetic bead covalently or non-covalently. In embodiments, the size of the oligonucleotides 102, 103 may be in the range of 100 to 200 base pairs, 120 to 180 base pairs, 130-170 base pairs, or the like. In embodiments, the oligonucleotides are characterized as oligomeric compounds in the form of single-stranded, double-stranded, circular, branched or hairpins and can contain structural elements such as internal or terminal bulges or loops. In embodiments, oligomeric double-stranded compounds can be two strands hybridized to form double-stranded compounds or a single strand with sufficient self-complementarity to allow for hybridization and formation of a fully or partially double-stranded compound. In embodiments, the oligonucleotides bound to the magnetic bead 101 includes a combination of primary oligonucleotides 102 and secondary oligonucleotides 103. In embodiments, one or more primary oligonucleotides 102 are bound to a magnetic bead 101, while one or more secondary oligonucleotides 103 are attached to the one or more primary oligonucleotides 102. In embodiments, a 5′ end of a secondary oligonucleotide 103 includes one or more reporter molecules 104 or labels. In embodiments, the reporter molecule 104 may be, for example, an enzyme, a fluorescent protein, a quantum dot, or any other type of reporter molecule which can be used directly and indirectly for spectroscopic, colorimetric, optical or electrochemical detection. For example, the reporter molecule may be, but not limited to, alkaline phosphatase, horseradish peroxidase (HRP), glucosidase, etc. In embodiments, the reporter molecule 104 may be linked covalently or non-covalently to the secondary oligonucleotide 103.

In a further embodiment, the secondary oligonucleotides 103 includes one or more portions or segments of DNA sequence 103A which may be complementary to the primary oligonucleotides 102. For example, the one or more portions or segments of DNA sequence complimentary to the primary oligonucleotides 102 may be at a 5′ and 3′ end of the secondary oligonucleotides 103. In embodiments, the secondary oligonucleotides 103 may also include a portion or segment of DNA sequence 103B which may not be complementary to the primary oligonucleotide 102, thereby forming a loop-like structure (including double stranded and single stranded regions of oligonucleotides). In embodiments, the non-complementary portion 103B of the secondary oligonucleotide 103 may be in a range between 10 to 100 base pairs. In embodiments, one or more non-complementary regions 103B of the secondary oligonucleotide 103 is predetermined, and/or complementary to one or more target nucleic acids present in the sample. This enables the device 100 to bind to the target nucleic acids in the sample, when brought in contact with the sample. In embodiments, the secondary oligonucleotide 103 binds to the target nucleic acid under normal physiological conditions. In an alternate embodiment, the secondary oligonucleotides 103 include 5 to 30 base pairs at 3′ end which is complementary to the primary oligonucleotides 102. Similarly, the secondary oligonucleotides 103 include 5 to 30 base pairs at the 5′ end which may be complementary to the primary oligonucleotide 102 to various degrees, while being fully complementary to the target nucleic acids present in the sample. In an embodiment, when the target nucleic acids in the sample bind to the single stranded region 103B of the secondary oligonucleotides 103, the binding may further extend down an outer end of the secondary oligonucleotide 103 replacing the primary oligonucleotide 102.

FIG. 2 illustrates a device 100 for detection of target nucleic acids, according to another embodiment. The device 100 is bound to one or more oligonucleotides 103. The oligonucleotides 103 are single stranded small fragments of nucleic acids such as DNA or RNA, ranging between 50 to 200 base pairs, 50 to 175 base pairs, or 50 to 160 base pairs, and the like. In embodiments, the oligonucleotides 103 are bound to or associated with the device covalently or non-covalently, for example, using a biotin-streptavidin link. Further, the oligonucleotides 103 are linked to a reporter molecule 104 at the 5′ end of the oligonucleotides. The reporter molecule 104 may be linked covalently or non-covalently with the oligonucleotides 103. In an embodiment, the oligonucleotides 103 may be chosen such that they are complementary to the target nucleic acids present in the sample.

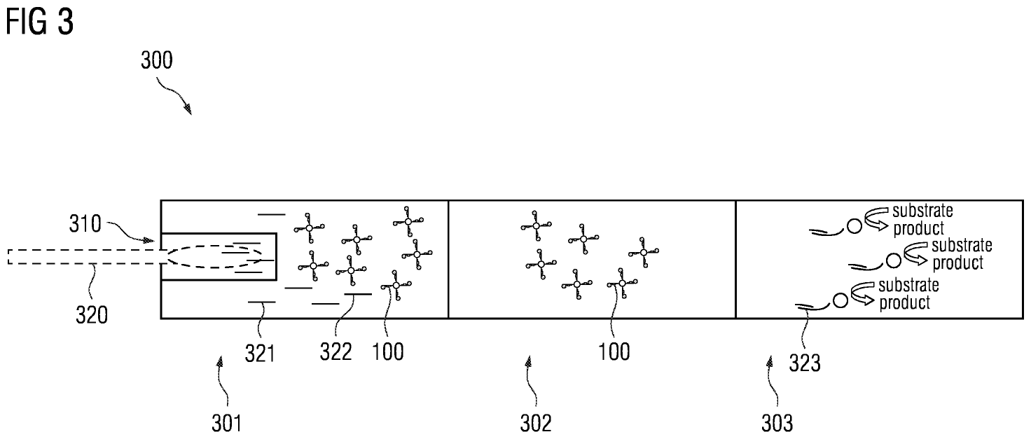

FIG. 3 illustrates an apparatus 300 for detection of target nucleic acids, according to an embodiment of the present disclosure. The apparatus 300 includes a first chamber 301 configured to receive the sample. In embodiments, apparatus 300 is characterized as a microfluidic device. For example, the sample may be obtained from an individual in a form of nasopharyngeal swab 320. The apparatus 300, therefore, may include an inlet 310 through which the nasopharyngeal swab 320 containing the sample may be inserted. Alternatively, the sample may also be body fluid obtained from the individual such as urine, sputum, etc. The sample can be introduced into the apparatus 300 through the inlet 310. In an embodiment, the first chamber 301 comprises a buffer and a plurality of device 100. The buffer may be a lysis buffer. The lysis buffer releases and stabilizes the target nucleic acids from pathogens present in the sample. Advantageously, this enables the device 100 to effectively bind with the target nucleic acids in the sample. The target nucleic acids bind with the oligonucleotides 103 in the device 100 to form a complex 323. In an embodiment, the apparatus 300 includes a first intermediate chamber 302 configured to receive the device 100 along with the complex 323 from the first chamber 301. In embodiments, the device 100 along with the complex 323 is subjected to heat in the first intermediate chamber 302. In embodiments, the various chambers are in fluid communication. The rise in temperature in the first intermediate chamber 302 may range between 2° C. to 50° C. Due to the heat, the complex 323 destabilizes from the device 100 and is separated. In an alternate embodiment, the first intermediate chamber 302 may comprise restriction enzymes specific to the complex 323 formed between the oligonucleotide 103 and the target nucleic acid present in the sample. The restriction enzymes may be specific to DNA-DNA hybrid or DNA-RNA hybrid formed between the oligonucleotide 103 and the target nucleic acids. Thus, the restriction enzymes cleave the hybrid/complex 323, thereby separating the complex 323 from the device 100. In another alternate embodiment, the first chamber 301 may be subjected to heat, or may include restriction enzymes, thereby separating the complex in the first chamber 301 itself.

The apparatus 300 further includes a second chamber 303 including a substrate capable of reacting with the reporter molecule 104 linked to the complex 323. The substrate may vary depending on the reporter molecule 104. For example, if the reporter molecule 104 is luciferase, the substrate in the second chamber 303 is luciferin. In another example, if the reporter molecule 104 is a quantum dot, light acts as a substrate to produce a glowing effect. Similarly, if the reporter molecule is alkaline phosphatase, phosphates act as a substrate to produce chemiluminescence. In embodiments, the reporter is selected from the group consisting of streptavidin β-galactosidase, substrate resorufin β-D-galactopyranoside, and combinations thereof. The second chamber 303 is configured to receive the separated complex 323 from the first chamber 301/first intermediate chamber 302. The reporter molecule 104 linked to the separated complex 323 reacts with the substrate to produce a product such as luminescence, fluorescence, etc. The amount of product generated is directly proportional to the amount of the target nucleic acids present in the sample.

In an alternate embodiment, the apparatus 300 may include a second intermediate chamber configured for performing amplification of the target nucleic acids that form a part of the separated complex 323. The second intermediate chamber may be configured to receive the separated complex 323 from the first chamber 301/first intermediate chamber 302 for the nucleic acid amplification process. Advantageously, the sensitivity of detection of target nucleic acids in the sample is increased by the amplification process.

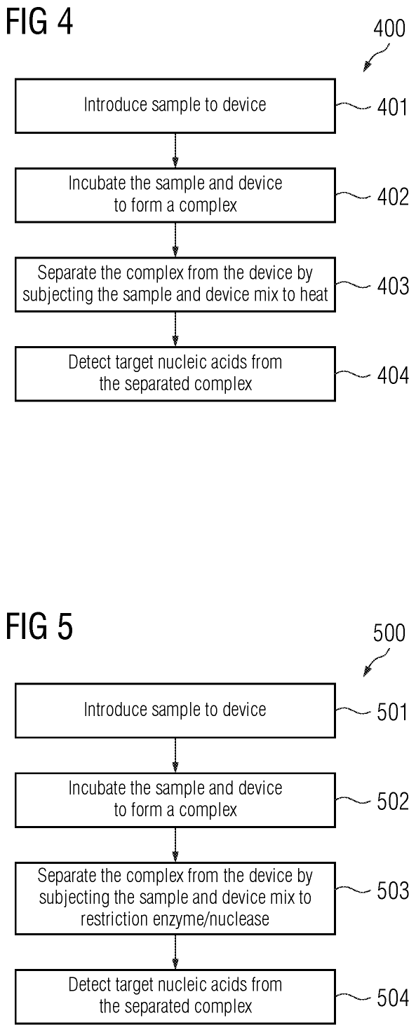

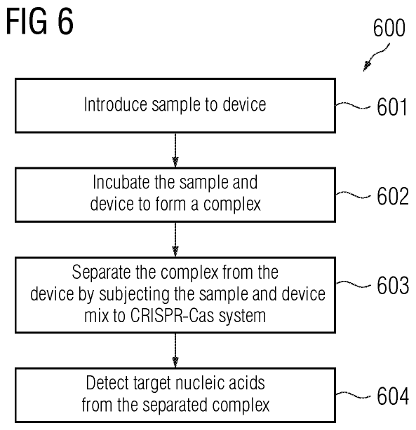

FIG. 4 illustrates a method 400 of detection of target nucleic acids in a sample, according to an embodiment of the present disclosure. At step 401, the sample is introduced to the device 100 such that the target nucleic acids present in the sample are brought in contact with the device 100. In the present embodiment, the device 100 is bound to one or more oligonucleotides, wherein the one or more oligonucleotides is a combination of primary oligonucleotide 102 and secondary oligonucleotide 103. The secondary oligonucleotide 103 is linked to the reporter molecule 104. At step 402, the sample and the device 100 are incubated to form a complex 323. The complex 323 includes target nucleic acids from the sample bound to the secondary oligonucleotides 103 of the device 100. At step 403, the complex 323 is separated from the device 100. In the present embodiment, once incubated, the sample and the device 100 mix is subjected to heat. The temperature of the mix is increased in a range of 2° C. and 50° C. The increase in temperature causes displacement of the secondary oligonucleotide 103 from the primary oligonucleotide 102. The secondary oligonucleotide 103 remains bound to the target nucleic acids in the sample. Further, at step 404 the target nucleic acids in the sample are detected from the separated complex 323. The detection is performed by introducing the separated complex 323 to a substrate specific to the reporter molecule 104. The reporter molecule 104 reacts with the substrate to generate a reaction product. The reaction product is quantified to detect the amount of target nucleic acids present in the sample. The quantity of the reaction product is directly proportional to the quantity of target nucleic acids present in the sample. The quantification of the reaction product may be performed using spectroscopic, optical and/or electrochemical methods.

FIG. 5 illustrates yet another embodiment of a method 500 of detecting target nucleic acids in a sample. At step 501, the sample is introduced to the device 100 such that the target nucleic acids present in the sample are brought in contact with the device 100. In the present embodiment, the device 100 is bound to one or more oligonucleotides 102, wherein the oligonucleotide 102 is single stranded small fragments of nucleic acids such as DNA or RNA, ranging between 50 to 200 base pairs. The oligonucleotide 102 is linked to the reporter molecule 104. At step 502, the sample and the device 100 are incubated to form a complex 323. The complex 323 includes the target nucleic acids from the sample bound to the oligonucleotides 102 of the device 100. At step 503, the complex 323 is separated from the device 100. In the present embodiment, once incubated, the sample and the device 100 mix is subjected to a restriction enzyme. The restriction enzyme may be specific to DNA/RNA hybrids wherein the oligonucleotide 102 is a DNA and the target nucleic acid is an RNA, or vice versa. Therefore, the restriction enzyme is configured to cleave the oligonucleotides 102 on the device 100 only if the oligonucleotides 102 are bound to the target nucleic acids in the sample. Alternatively, the sample and the device 100 mix may be subjected to a nuclease enzyme. The nuclease enzyme may be a DNA/DNA nuclease or a DNA/RNA nuclease.

Once cleaved, the complex 323 is separated from the device 100. Further, at step 504 the target nucleic acids in the sample are detected from the separated complex 323. The detection is performed by introducing the separated complex 323 to a substrate specific to the reporter molecule 104. The reporter molecule 104 reacts with the substrate to generate a reaction product. The reaction product is quantified to detect the amount of target nucleic acids present in the sample. The quantity of the reaction product is directly proportional to the quantity of target nucleic acids present in the sample. The quantification of the reaction product may be performed using spectroscopic, optical and/or electrochemical methods.

In an alternative embodiment illustrated in FIG. 6, the separation of the complex 323 from the device 100 is achieved by introducing a CRISPR-Cas system. CRISPR-Cas system is widely used in gene editing and is adapted to many diagnostic detection workflows. The CRISPR-Cas system includes a single stranded guide RNA (sgRNA) and a CRISPR-associated (Cas) nuclease enzyme. The sgRNA is a nucleotide sequence configured to recognize the target nucleic acids in the complex 323 and directs the Cas nuclease for cleaving. The sgRNA may include CRISPR RNA (crRNA), a 17-20 nucleotide sequence complimentary to the target DNA and a tracr-RNA which serves as a binding scaffold for the Cas nuclease. The Cas nuclease may be for example, Cas12a and/or Cas 13, which when they detect double stranded DNA or a single stranded RNA respectively with the aid of sgRNA, induces rampant cleavage of surrounding oligonucleotides. According to FIG. 6, at step 601, the sample is introduced to the device 100 to enable contact between the device 100 and the target nucleic acids present in the sample. At step 602, the sample and the device 100 are incubated to form a complex 323. At step 603, CRISPR-Cas system is introduced to induce separation of the complex 323 from the device 100. The sgRNA binds to the complex 323 and the Cas nuclease cleaves the complex 323 from the device 100. At step 604, the target nucleic acids are detected from the separated complex 323. The reporter molecule 104 reacts with the substrate to generate a reaction product. For example, if the reporter molecule is a fluorophore and a quencher, Cas nuclease induced cleavage releases the fluorophore resulting in fluorescence. The fluorescence may be quantified to detect the amount of target nucleic acids in the sample. The quantity of the reaction product is directly proportional to the quantity of target nucleic acids present in the sample.

The above embodiment enables detection of target nucleic acids faster and more accurately. Advantageously, the embodiment involves minimal or no require sample preparation, thereby reducing turn-around time. Additionally, the embodiment uses simple instrumentation with magnetic actuation of reaction mix, making it suitable for point-of-care applications. Yet another advantage of the embodiment is that the method is highly sensitive and does not require complex procedures such as nucleic acid purification or amplification for the detection process. Therefore, the embodiment enables detection of nucleic acids from crude samples, without involving wash-steps. This makes the method a homogenous method amenable to be under in a point-of-care instrument.

### Targets

Applicable targets in embodiments of the present disclosure can be derived from virtually any source. Typically, the targets will be nucleic acid molecules from a biological sample. Target nucleic acids may be relatively long (typically thousands of bases), or short having 50-1000, 50-500, or 50 to 150 base pairs. Targets may be obtained from samples. Samples can be obtained from a single source (e.g., one patient or tissue) or from multiple sources. Samples may be obtained from a plurality of subjects, tissues, etc. In some embodiments, samples are obtained from a single subject at multiple time points and the differences between the time points ascertained.

In some embodiments of the present disclosure, targets are capped by amount. For example, the amount of a particular target may be capped to be less than 10 μg, 5 μg, 1 μg. 500 ηg, 100 ηg, 90 ηg, 80 ηg, 70 ηg, 60 ηg, 50 ηg, 40 ηg, 30 ηg, 20 ηg, 10 ηg, 5 ηg, 1 ηg, 500 picograms, etc. The target cap appropriate for a given application may be influenced by a variety of factors, including sample type, sample number, sample amount, or sequencing platform. Those of skill in the art will appreciate that the cap may be set as necessary.

### Certain Embodiments

Certain embodiments of the present disclosure include:

Embodiment 1: A device (100) for detection of target nucleic acids in a sample, the device (100) including: a magnetic bead (101); one or more oligonucleotides (102, 103) bound to the magnetic bead (101); and at least one reporter molecule (104) linked to the one or more oligonucleotides (103).

Embodiment 2: A device (100) for detection of target nucleic acids in a sample, the device (100) including: a magnetic bead (101); one or more oligonucleotides (102, 103) bound to the magnetic bead (101); and at least one reporter molecule (104) linked to the one or more oligonucleotides (103), wherein the one or more oligonucleotides (103) comprises at least one portion of nucleotides complimentary to the target nucleic acids present in the sample.

Embodiment 3: A device (100) for detection of target nucleic acids in a sample, the device (100) including: a magnetic bead (101); one or more oligonucleotides (102, 103) bound to the magnetic bead (101); and at least one reporter molecule (104) linked to the one or more oligonucleotides (103), wherein the one or more oligonucleotides is a combination of a primary oligonucleotide (102) linked to a secondary oligonucleotide (103), wherein at least one portion of the secondary oligonucleotide (103) is complimentary to the primary oligonucleotide (102).

Embodiment 3: A device (100) for detection of target nucleic acids in a sample, the device (100) including: a magnetic bead (101); one or more oligonucleotides (102, 103) bound to the magnetic bead (101); and at least one reporter molecule (104) linked to the one or more oligonucleotides (103), wherein the one or more oligonucleotides is a combination of a primary oligonucleotide (102) linked to a secondary oligonucleotide (103), wherein at least one portion of the secondary oligonucleotide (103) is complimentary to the primary oligonucleotide (102), and wherein the at least one reporter molecule (104) is linked to the secondary oligonucleotide (103).

Embodiment 4: A device (100) for detection of target nucleic acids in a sample, the device (100) including: a magnetic bead (101); one or more oligonucleotides (102, 103) bound to the magnetic bead (101); and at least one reporter molecule (104) linked to the one or more oligonucleotides (103), wherein the one or more oligonucleotides is a combination of a primary oligonucleotide (102) linked to a secondary oligonucleotide (103), wherein at least one portion of the secondary oligonucleotide (103) is complimentary to the primary oligonucleotide (102), and wherein the at least one reporter molecule (104) is linked to the secondary oligonucleotide (103), wherein the secondary oligonucleotide (103) comprises at least one portion of nucleotides complimentary to the target nucleic acids present in the sample.

Embodiment 5: A device (100) for detection of target nucleic acids in a sample, the device (100) including: a magnetic bead (101); one or more oligonucleotides (102, 103) bound to the magnetic bead (101); and at least one reporter molecule (104) linked to the one or more oligonucleotides (103), wherein the reporter molecule (104) is one of an enzyme, a fluorescent protein, or a quantum dot . . . .

Embodiment 6: A method (400) of detecting target nucleic acids in a sample, the method (400) including: introducing the sample to a device (100), wherein the device (100) comprises a magnetic bead (101), one or more oligonucleotides (102, 103) bound to the magnetic bead and at least one reporter molecule (104) linked to the one or more oligonucleotides (102, 103); incubating the sample and the device (100) to form a complex (323), wherein the complex (323) comprises the target nucleic acids from the sample bound to the one or more oligonucleotides (103) in the device (100), wherein the one or more oligonucleotides (103) comprise at least one portion of oligonucleotides (103B) complimentary to the target nucleic acids present in the sample; separating the complex (323) from the device (100); and detecting the target nucleic acids in the sample from the separated complex (323). In embodiments, Embodiment 5 further includes, wherein separating the complex (323) from the device (100) comprises subjecting the complex to heat, wherein the heat destabilizes the complex (323) from the device (100). In embodiments, separating the complex (323) from the device (100) comprises subjecting the device (100) to a restriction enzyme, wherein the restriction enzyme cleaves the complex (323) from the device (100). In embodiments, the methods of the present disclosure are characterized as homogenous, no-wash, or combinations thereof.

Embodiment 7: An apparatus (300) for detection of target nucleic acids in a sample, the apparatus (300) including: a first chamber (301) configured to receive the sample, wherein the first chamber (301) comprises a buffer, wherein the first chamber (301) further comprises a plurality of device (100), wherein the device (100) comprises a magnetic bead (101), one or more oligonucleotides (102, 103) bound to the magnetic bead (101) and at least one reporter molecule (104) linked to the one or more oligonucleotides (103); and a second chamber (303) configured to receive the sample and the plurality of the device (100), wherein the second chamber (303) comprises a substrate capable of reacting with a reporter molecule (104) linked to the one or more oligonucleotides (103) in the device (100). In embodiments. Embodiment 7 includes a second chamber (303) further includes a restriction enzyme, wherein the restriction enzyme is capable of cleaving the one or more oligonucleotides (103) from the device (100).

Embodiment 8: A use of an apparatus (300) as described herein for detection of target nucleic acids in a sample.

Embodiment 9: A kit include a device, apparatus of the present disclosure. In embodiments the kit includes a swab, solution, buffer, collection tube, and the like, and combinations thereof.

### Example I (Prophetic Example)

A patient presents with an infection. A specimen or biological sample is taken from the patient. A device of the present disclosure is mixed the biological sample. The device is suitable for and configured for detecting one or more target nucleic acids in the specimen or sample. The device includes a magnetic bead and one or more oligonucleotides bound to the magnetic bead. The device includes at least one reporter molecule or label linked to the one or more oligonucleotides, wherein the more oligonucleotides are preselected in length, and chemical composition. The one or more oligonucleotides are double stranded and include a loop structure, wherein the loop structure includes a first single stranded nucleic acid segment and a second single stranded nucleic acid segment, wherein one of the first or second single stranded nucleic acid segments is characterized as an analyte specific binding segment. The device is contacted with a specimen including an analyte that binds the analyte specific binding segment when contacted under suitable conditions such as normal physiological conditions to form a complex. The complex is isolated or purified from the specimen. Further analysis provides information on the target and pathogen associated therewith.

### Example II (Prophetic Example)

A cartridge or apparatus of the present disclosure is used for the detection of Neisseria gonorrhoeae (NG). A patient swab is obtained and directly inserted into the cartridge. The bacteria on the swab is lysed when dipped in the lysis solution with additional heating or no heating. The lysed sample is incubated with the beads or device of the present disclosure. Referring to FIG. 1, a portion of 102 is designed specific to NG. The secondary oligo is also designed to interact with 102 at the same region as the NG but via complementarity at 5′ and 3′ends. The secondary oligo is linked to reporter enzyme such as streptavidin β-galactosidase (SβG). When the temperature is increased, in the presence of NG, the bacterial genome interacts with 102 as it has 100% complementary sequence and displaces the secondary oligo. Thus, the secondary oligo remains in the supernatant when the beads are magnetized. The supernatant is then separated and checked for the activity of SβG using resorufin β-D-galactopyranoside. The signal is directly proportional to the presence of the NG. In the absence of NG, the secondary oligo region (103A) binds back or remains bound to the 102 and is not released into the supernatant. Thus, when the beads are magnetized, and supernatant is separated and tested for SβG activity no signal is observed.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials, and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may give effect to numerous modifications thereto and changes may be made without departing from the scope and spirit of the invention in its aspects.

While the foregoing is directed to embodiments of the present disclosure, other and further embodiments of the disclosure may be devised without departing from the basic scope thereof.

What is claimed is:

1. A device (100) for detection of target nucleic acids in a sample, the device (100) comprising:
a magnetic bead (101);
one or more oligonucleotides (102, 103) bound to the magnetic bead (101); and
at least one reporter molecule (104) linked to the one or more oligonucleotides (103).

2. The device (100) according to claim 1, wherein the one or more oligonucleotides (103) comprises at least one portion of nucleotides complimentary to the target nucleic acids present in the sample.

3. The device (100) according to claim 1, wherein the one or more oligonucleotides is a combination of a primary oligonucleotide (102) linked to a secondary oligonucleotide (103), wherein at least one portion of the secondary oligonucleotide (103) is complimentary to the primary oligonucleotide (102).

4. The device (100) according to claims 1 and 3, wherein the at least one reporter molecule (104) is linked to the secondary oligonucleotide (103).

5. The device (100) according to claim 3, wherein the secondary oligonucleotide (103) comprises at least one portion of nucleotides complimentary to the target nucleic acids present in the sample.

6. The device (100) according to claim 1, wherein the reporter molecule (104) is one of an enzyme, a fluorescent protein, or a quantum dot.

7. A method (400) of detecting target nucleic acids in a sample, the method (400) comprising:
introducing the sample to a device (100), wherein the device (100) comprises a magnetic bead (101), one or more oligonucleotides (102, 103) bound to the magnetic bead and at least one reporter molecule (104) linked to the one or more oligonucleotides (102, 103);
incubating the sample and the device (100) to form a complex (323), wherein the complex (323) comprises the target nucleic acids from the sample bound to the one or more oligonucleotides (103) in the device (100), wherein the one or more oligonucleotides (103) comprise at least one portion of oligonucleotides (103B) complimentary to the target nucleic acids present in the sample;
separating the complex (323) from the device (100); and
detecting the target nucleic acids in the sample from the separated complex (323).

8. The method (400) according to claim 7, wherein separating the complex (323) from the device (100) comprises subjecting the complex to heat, wherein the heat destabilizes the complex (323) from the device (100).

9. The method (400) according to claim 7, wherein separating the complex (323) from the device (100) comprises subjecting the device (100) to a restriction enzyme, wherein the restriction enzyme cleaves the complex (323) from the device (100).

10. The method (400) according to claim 7, wherein separating the complex (323) from the device (100) comprises subjecting the device (100) to a Cas nuclease enzyme, wherein the Cas nuclease enzyme cleaves the complex (323) from the device (100).

11. The method (400) according to claim 7, wherein detecting the target nucleic acids in the sample from the separated complex (323) comprises:
introducing a substrate specific to the reporter molecule (104) linked to the one or more oligonucleotides (103) in the complex (323) to generate a reaction product; and
quantifying the reaction product, wherein the quantity of the reaction product is proportional to the amount of the target nucleic acid present in the sample.

12. An apparatus (300) for detection of target nucleic acids in a sample, the apparatus (300) comprising:
a first chamber (301) configured to receive the sample, wherein the first chamber (301) comprises a buffer, wherein the first chamber (301) further comprises a plurality of device (100), wherein the device (100) comprises a magnetic bead (101), one or more oligonucleotides (102, 103) bound to the magnetic bead (101) and at least one reporter molecule (104) linked to the one or more oligonucleotides (103); and
a second chamber (303) configured to receive the sample and the plurality of the device (100), wherein the second chamber (303) comprises a substrate capable of reacting with a reporter molecule (104) linked to the one or more oligonucleotides (103) in the device (100).

13. The apparatus (300) according to claim 12, wherein the second chamber (303) further comprises a restriction enzyme, wherein the restriction enzyme is capable of cleaving the one or more oligonucleotides (103) from the device (100).

14. The apparatus (300) according to claim 12, wherein the second chamber (303) further comprises a CRISPR-Cas system, wherein the Cas enzyme is capable of cleaving the one or more oligonucleotides (103) from the device (100).

15. A use of an apparatus (300) as claimed in claims 12, 13 and 14 for detection of target nucleic acids in a sample.
